(19) United States
(12) Patent Application Publication
(10) Pub. No.: US 2005/0124914 A1
(43) Pub. Date: Jun. 9, 2005

(54) Medical instrument

(76) Inventors: Paul Dicarlo, Middleboro, MA (US); David Cooke, Groveland, MA (US)

Correspondence Address: FISH & RICHARDSON PC, 225 FRANKLIN ST, BOSTON, MA, 02110, US

(21) Appl. No.: 10/728,248

(22) Filed: Dec. 4, 2003

(51) Int. Cl. A61B 17/32; A61B 10/00

(52) U.S. Cl. 600/567; 606/167

(57) ABSTRACT

Medical instruments and methods of using the instruments are described. In some embodiments, a medical instrument includes a housing, a stylet having a portion in the housing, and a cannula having a portion in the housing. The stylet has a portion in the housing and is movable between a first extended position and a first retracted position. The stylet is configured to rotate when moved from the first retracted position to the first extended position. The cannula coaxially receives the stylet and is movable between a second extended position and a second retracted position.

### TECHNICAL FIELD

The invention relates to medical instruments, such as a biopsy needle instrument.

### BACKGROUND

A biopsy needle instrument can be used to obtain a tissue specimen for microscopic examination, e.g., to determine malignancy, while preferably subjecting the patient to the least trauma. In some embodiments, such instruments can have of a long, thin probe, called a stylet, within a close-fitting hollow needle, called a cannula. The stylet has a notch into which tissue can prolapse when the stylet enters the tissue.

During use, a firing device first projects the stylet into tissue, followed immediately by the cannula. As the cannula slides over the stylet, the cannula severs tissue that has prolapsed into the notch of the stylet from the surrounding mass, and captures the prolapsed tissue as a specimen within the notch. The instrument can then be withdrawn and the piece of tissue removed from the stylet.

### SUMMARY

The invention relates to medical instruments.

In one aspect, the invention features a medical instrument including a housing having a proximal end and a distal end; a stylet having a portion in the housing, the stylet being movable between a first extended position and a first retracted position, the stylet being configured to rotate when moved from the first retracted position to the first extended position; and a cannula coaxially receiving the stylet and having a portion in the housing, the cannula being movable between a second extended position and a second retracted position.

Embodiments may include one or more of the following features. The instrument includes a stylet block attached to a proximal end of the stylet and mounted inside the housing. The stylet block includes a first part inside the housing, the first part being moveable between an extended position and a retracted position; and a second part attached to the proximal end of the stylet, the second part being rotatably engaged with the first part and being able to rotate relative to an axis of the stylet. The instrument further includes a stylet spring capable of moving the stylet from the first retracted position to the first extended position; and a cannula spring capable of moving the cannula from the second retracted position to the second extended position. The instrument further includes a first pivoting latch capable of retaining the stylet in a predetermined position when the stylet is in the first retracted position; and a second pivoting latch capable of retaining the cannula in a predetermined position when the cannula is in the second retracted position.

The housing can include a semi-cylindrical portion defining a track configured to engage with the second part. The second part can include a projection in contact with a track associated with the housing, the projection and track capable of cooperating to axially rotate the second part and the attached stylet when the stylet is moved between the first extended position and the first retracted position. The track can be molded into the interior side of the housing. The track can be configured to provide unidirectional or multidirectional rotation to the stylet.

The stylet can include a notch with a sharpened leading edge. The stylet can include a notch having two openings and/or a ramped surface. The stylet can include an opening opposing the notch.

In another aspect, the invention features a method of using a medical instrument. The method includes moving a stylet from a first position to a second position; simultaneously rotating the stylet along an axis of the stylet; and moving a cannula over the stylet.

Embodiments may include one or more of the following features. The stylet is oscillated along the axis. The method further includes collecting a sample in a notch of the stylet. Removal of the sample from the notch is achieved by inserting an object through an opening located in the notch. The method further includes removing the sample over an inclined portion of the notch. The stylet is rotated in one direction or multiple directions.

Other aspects and features of the invention will be apparent from the description of the preferred embodiments thereof and from the claims.

### DESCRIPTION OF DRAWINGS

FIGS. 1A and 1B are schematic drawings of a medical instrument having a stylet and a cannula in their retracted positions and extended positions, respectively.

FIG. 2 is a perspective view of a stylet attached to a stylet block.

FIGS. 3A and 3B are perspective views of the stylet and attached stylet block of FIG. 2 with an associated portion of a housing including a track.

FIG. 4 is a schematic view of a track.

FIG. 5A is a side view of a stylet notch; FIG. 5B is a cross-sectional view of the stylet notch of FIG. 5A, taken along line 5B-5B; and FIG. 5C is a cross-sectional view of another embodiment of a stylet notch.

FIG. 6A is a side view of a stylet notch; and FIG. 6B is a cross-sectional view of the stylet notch of FIG. 6A, taken along line 6B-6B.

FIG. 7 is a side view of a stylet notch.

FIG. 8A is a side view of a stylet notch; and FIG. 8B is a cross-sectional view of the notch of FIG. 8A, taken along line 8B-8B.

### DETAILED DESCRIPTION

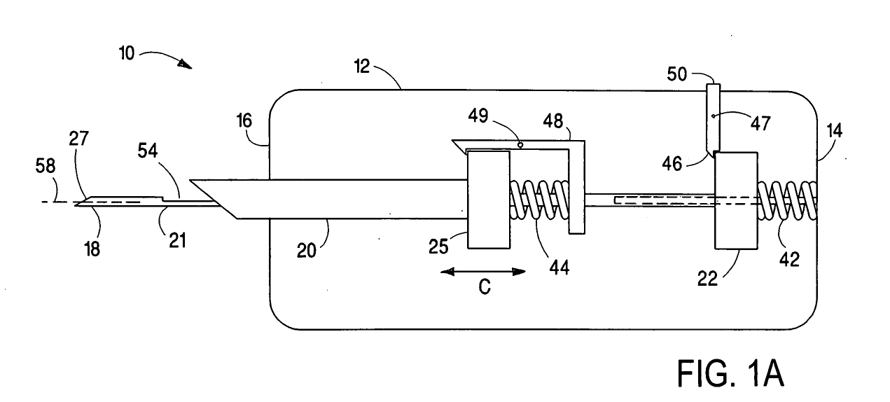

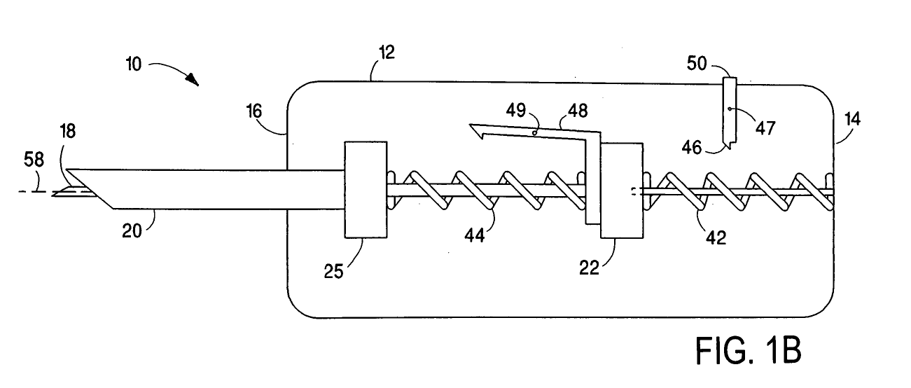

Referring to FIGS. 1A and 1B, a medical instrument 10 (as shown, a needle biopsy device) includes a housing 12, a stylet 18, and a cannula 20 coaxially receiving the stylet. Housing 12 has a proximal end 14 and a distal end 16. At its distal end 27, stylet 18 is configured to penetrate tissue and includes a cupped notch 54 configured to cut and to collect a tissue sample. At its distal end 21, cannula 20 is configured to sever tissue that has prolapsed into notch 54. Both stylet 18 and cannula 20 have portions in housing 12 and can be moved between retracted positions as shown in FIG. 1A and extended positions as shown in FIG. 1B. During use, stylet 18 and cannula 20 are loaded or cocked to their retracted positions, ready to be triggered. When stylet 18 and cannula 20 are triggered, they rapidly move distally to their extended positions, e.g., to collect a tissue specimen that has prolapsed into notch 54 of the stylet.

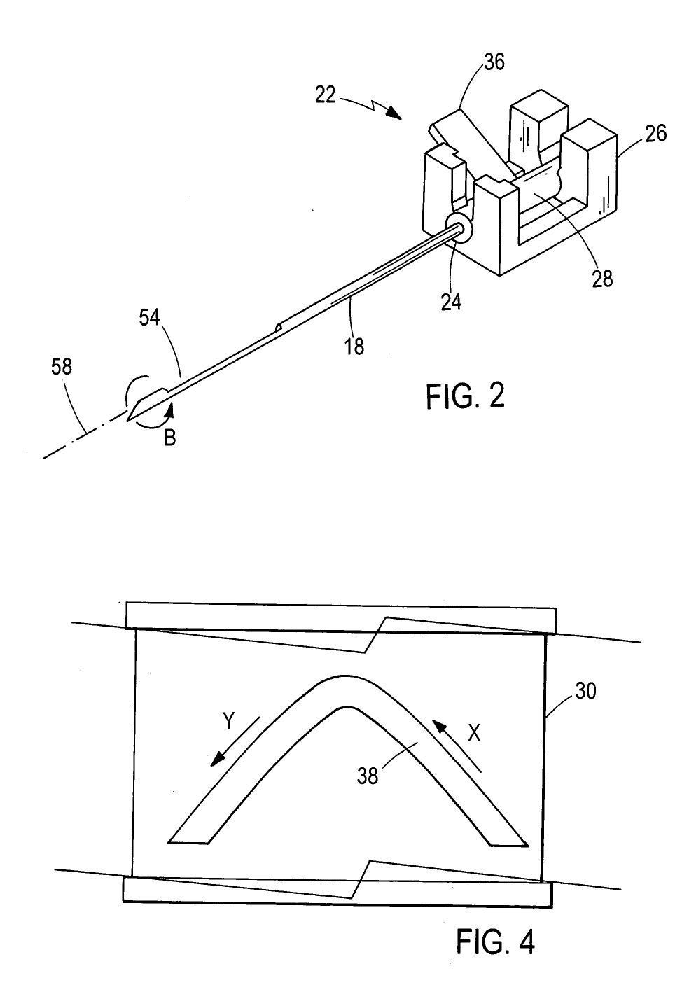

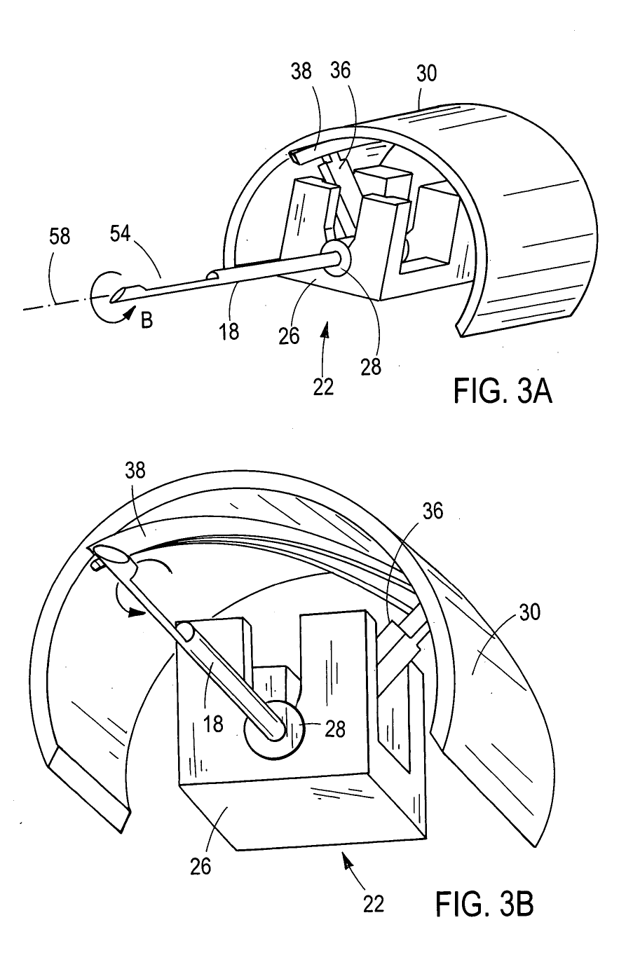

In particular, during use, stylet 18 is configured to rotate about its longitudinal axis 58 when moved from its retracted position to its extended position. The rotational motion of stylet 18 can enhance its cutting action, thereby enhancing the performance of instrument 10. Referring to FIGS. 2, 3A and 3B, at its proximal end 24, stylet 18 is attached to a stylet block 22. Stylet block 22 includes a first, outer part 26 and a second, inner part 28 (as shown, a generally tubular structure). Inner part 28 is connected to stylet 18 and can rotate within outer part 26, about the longitudinal axis 58 of the stylet. More specifically, inner part 28 includes a projection or an arm 36 extending radially outward from the inner part and engaging with a track 38. As shown in FIGS. 3A and 3B, track 38 is an elongated channel defined in a curved, semi-cylindrical member 30 connected to housing 12. Track 38 extends helically in a direction (A) parallel to axis 58 so that as stylet block 22 is propelled distally along direction A during use, projection 36 travels along the track and rotates inner part 28 and stylet 18 (arrow B). Track 38 can be extend such that projection 36 travels from greater than zero degree to about 175 degrees relative to a starting position. The degree of travel of projection 36 can be greater than or equal to about 0°, 30°, 60°, 90°, 120°, or 150°; and/or less than or equal to about 175°, 150°, 120°, 90°, 60°, or 30°, relative to the starting position.

In other embodiments, track 38 is configured to rotate stylet 18 in more than one direction. Referring to FIG. 4, track 38 extends along member 30 in a first direction X and then changes to a second direction Y, as shown, transverse to direction X. As a result, as projection 36 travels along track 38, stylet 18 rotates in a first direction and then in a second direction, e.g., clockwise and then counter-clockwise, thereby imparting an oscillating and rotating motion to the stylet. Track 38 can change direction multiple times to impart more oscillations. In some embodiments, track 38 is defined by the interior surface of housing 12, e.g., by injection molding.

Referring again to FIGS. 1A and 1B, cannula 20 is a hollow sheath that slidably receives stylet 18. From distal end 21, cannula 20 extends into housing 12 where the cannula is attached to a longitudinally movable (arrow C) cannula block 25.

Still referring to FIGS. 1A and 1B, instrument 10 further includes components that retain stylet 18 and cannula 20 in their retracted positions, and components that propel the stylet and the cannula to their extended positions. To hold cannula 20 and stylet 18, instrument 10 includes, respectively, a cannula latch 48 mounted pivotally to housing 12 at point 49 and a stylet latch 46 mounted pivotally to the housing at point 47 (FIG. 1A). Stylet latch 46 extends to the exterior of housing 12 to form a trigger 50 capable of firing instrument 10 during use, as described below. Both latches 48 and 46 are capable of pivoting about their respective attachment points to release the stylet or the cannula from engagement. To propel cannula 20 and stylet 18, instrument 10 includes, respectively, a cannula spring 44 located proximal of cannula block 25, and a stylet spring 42 located proximal to stylet block 22.

Other features and embodiments of needle biopsy devices are described in commonly-assigned U.S. Ser. No. 10/300,249, filed Nov. 20, 2002; and U.S. Ser. No. 10/300,512, filed Nov. 20, 2002, both hereby incorporated by reference. Examples of suitable stylet 18 and cannula 20 configurations are exemplified by the ASAP™ Automated Biopsy System having a Delta Cut® needle or a Channel Cut® needle (available from Boston Scientific Corp., Natick, Mass.), and described in Chu, U.S. Pat. No. 5,989,196, hereby incorporated by reference.

In operation, cannula 20 and stylet 18 are loaded (e.g., moved proximally and retained in their retracted positions) and subsequently fired (e.g., released and propelled distally). More specifically, cannula 20 and cannula block 25 are first moved proximally until the cannula block engages with and is held by cannula latch 48 (FIG. 1A). Cannula spring 44 is compressed between cannula block 25 and cannula latch 48. Next, stylet 18 and stylet block 22 are moved proximally until the stylet block engages with and is held by stylet latch 46. Stylet block 22 compresses stylet spring 42. Instrument 10 is loaded and ready to be fired.

To fire instrument 10, distal end 27 of stylet 18 is placed adjacent to a target area, and trigger 50 is actuated. Sliding trigger 50 proximally pivots stylet latch 46 about point 47 and disengages the stylet latch from stylet block 22. Upon disengagement, stylet block 22 and stylet 18 are propelled distally by the spring force of stylet spring 42, which allows the stylet to penetrate the targeted area, e.g., tissue. As stylet 18 translates distally, projection 36 of stylet block 22 travels along track 38 (e.g., distally and spirally), thereby imparting rotational motion to the stylet 18. Stylet block 22 then strikes cannula latch 48, which causes the cannula latch to pivot about point 49 and to disengage from stylet block 25. Upon disengagement, cannula block 25 and cannula 20 are propelled distally by the spring force of spring 44, which allows the cannula to slide over stylet 18 and to sever a specimen that has prolapsed into notch 54 of stylet 18.

Instrument 10 can then be withdrawn from the targeted area. The specimen can be removed from notch 54 by first retracting cannula 20 and cannula block 25 proximally. The specimen can be placed on a slide or in a preservative solution. If desired, stylet 18 can be retracted to load instrument 10 and to collect another specimen.

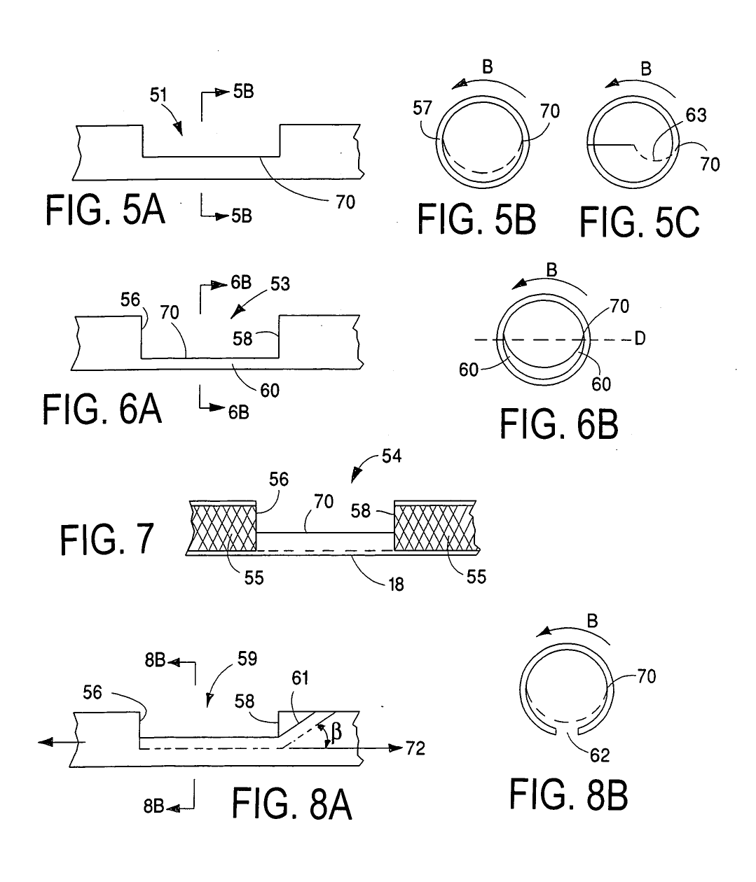

In other embodiments, other configurations or designs of notch 54 can be used. For example, referring to FIGS. 5A and 5B, a notch 51 is defined in part by a leading edge 70 and a trailing edge 57. Leading edge 70, which is used to cut tissue, can be relieved to sharpen the edge and to enhance cutting. The relief can be an off-center cut using, for example, a ball end mill to produce a small undercut or chamfer that forms a facet to serve as a cutting edge. The cutting edge can have a thickness of about 0.002 inch or less. In some cases, notch 51 can be formed by broaching the notch from a stylet. Broaching allows minimal material to be removed per cut, thereby reducing (e.g., minimizing) induced stresses and material deformation. The cupped portion of the notch can be formed by milling with a ball end milling apparatus that has a diameter larger than the stylet to form a sharpened edge (e.g., edge 70). The edge can be polished to remove any burrs and further enhance sharpness. In other embodiments, referring to FIG. 5C, the cupped portion 63 of a notch extends to less than the diameter or width (as shown, half) of the notch. The remaining cross-sectional portion of the notch can enhance the strength of the stylet.

Alternatively or in addition, a substantial portion of material of stylet 18 can be removed in the vicinity of a notch to enhance (e.g., increase) the size of a specimen that can be collected. Referring to FIGS. 6A and 6B, a notch 53 is defined by distal edge 56, a proximal edge 58, and a sidewall 60. Sidewall 60 can be lowered to increase to volume of notch 53. In some embodiments, the top of sidewall 60 is formed anywhere from about 15% of the outer diameter of stylet 18 below the center line (D) to about 15% of the outer diameter of the stylet above the center line (D). For example, the top of sidewall 60 can be about 10% or 5% of the stylet O.D. below or above the center line (D).

In some cases, stylet 18 can be reinforced to enhance its strength. Referring to FIG. 7, stylet 18 can include solid plugs 55 located proximally and/or distally to notch 54. Plugs 55 can be made of, for example, stainless steel rods welded or soldered to a tubular stylet, to enhance the rigidity of stylet 18, such as in embodiments in which the sidewalls are lowered. Plugs 55 also provide other methods of making stylet 18. For example, a notch can be formed in a hollow tube, which is subsequently reinforced with plugs 55 to form stylet 18. By starting with a hollow tube, vis-a-vis a solid tube, less material need to be removed to form the notch, thereby reducing waste, manufacturing time, and cost.

A stylet notch can also include features that enhance removal of a specimen. Referring to FIGS. 8A and 8B, a notch 59 includes a ramped surface 61 and a bottom slot 62 (i.e., notch 59 has a second opening other than the opening that defines the notch). A specimen in notch 59 can be removed by sliding the specimen up ramped surface 61. Alternatively or in addition, an object, such as a probe, can be inserted through slot 62 to dislodge the specimen from notch 59. In some embodiments, the ramped surface is inclined (β) about 5 to about 80 degrees relative to longitudinal axis 72. Ramped surface 61 can be formed at the distal and/or proximal end of a notch. Slot 62 can extend the entire length of a notch or only a portion thereof. Slot 62 can be of any shape, e.g., rectangular, oval, polygonal, etc. In some cases, ramped surface 61 defines the proximal portion of notch 59, i.e., the notch does not include proximal edge 58. A ramped surface and/or an opening can be combined with any of the embodiments of notches described above.

In some embodiments, housing 12 can be made of different materials, e.g., to enhance the grip or “feel” of instrument 10. For example, housing 12 can be formed of materials with different hardness, e.g., a core of relatively hard material and an outer layer of relatively soft material. The outer layer can be a foamy material, such as a urethane, to enhance the grip and/or to absorb vibrations from the firing of instrument 10. Housing 12 can be formed with two or more different materials.

The components of instrument 10 (e.g., housing 12, latches 46 and 48, stylet block 22, or cannula block 25) described above can be formed by conventional injection molding techniques, e.g., of polycarbonate and/or ABS. Stylet 18, cannula 20, and springs can be formed of stainless steel.

Terms such as “side” or “bottom” are used to describe embodiments as shown in the orientation of the figures and not intended to be limiting.

Other embodiments are within the claims.

1. A medical instrument, comprising:
a housing having a proximal end and a distal end;
a stylet having a portion in the housing, the stylet being movable between a first extended position and a first retracted position, the stylet being configured to rotate when moved from the first retracted position to the first extended position; and
a cannula coaxially receiving the stylet and having a portion in the housing, the cannula being movable between a second extended position and a second retracted position.

2. The instrument of claim 1, further comprising a stylet block attached to a proximal end of the stylet and mounted inside the housing.

3. The instrument of claim 2, wherein the stylet block comprises:
a first part inside the housing, the first part being moveable between an extended position and a retracted position; and
a second part attached to the proximal end of the stylet, the second part being rotatably engaged with the first part and being able to rotate relative to an axis of the stylet.

4. The instrument of claim 3, wherein the housing comprises a semi-cylindrical portion defining a track configured to engage with the second part.

5. The instrument of claim 1, wherein the second part comprises:
a projection in contact with a track associated with the housing, the projection and track capable of cooperating to axially rotate the second part and the attached stylet when the stylet is moved between the first extended position and the first retracted position.

6. The instrument of claim 5, wherein the track is molded into the interior side of the housing.

7. The instrument of claim 5, wherein the track is configured to provide unidirectional rotation to the stylet.

8. The instrument of claim 5, wherein the track is configured to provide multidirectional rotation to the stylet.

9. The instrument of claim 1, further comprising:
a stylet spring capable of moving the stylet from the first retracted position to the first extended position; and
a cannula spring capable of moving the cannula from the second retracted position to the second extended position.

10. The instrument of claim 1, further comprising:
a first pivoting latch capable of retaining the stylet in a predetermined position when the stylet is in the first retracted position; and
a second pivoting latch capable of retaining the cannula in a predetermined position when the cannula is in the second retracted position.

11. The instrument of claim 1 wherein the stylet comprises a notch with a sharpened leading edge.

12. The instrument of claim 1, wherein the stylet comprises a notch having two openings.

13. The instrument of claim 1, wherein the stylet comprises a notch with a ramped surface.

14. The instrument of claim 13, wherein the stylet further comprises an opening opposing the notch.

15. A method of using a medical instrument, the method comprising:
moving a stylet from a first position to a second position;
simultaneously rotating the stylet along an axis of the stylet; and
moving a cannula over the stylet.

16. The method of claim 15, further comprising oscillating the stylet along the axis.

17. The method of claim 15, further comprising collecting a sample in a notch of the stylet.

18. The method of claim 17, further comprising removing the sample from the notch by inserting an object through an opening located in the notch.

19. The method of claim 17, further comprising removing the sample over an inclined portion of the notch.

20. The method of claim 15, comprising rotating the stylet in one direction.

21. The method of claim 15, comprising rotating in multiple directions.
